(19) United States
(12) Patent Application Publication
(10) Pub. No.: US 2024/0138416 A1
(43) Pub. Date: May 2, 2024

(54) Microbial Compositions For Preserving Healthy Soils And Restoring Degraded Soils

(71) Applicant: LOCUS SOLUTIONS IPCO, LLC, Solon, OH (US)

(72) Inventors: Paul ZORNER, Encinitas, CA (US); Sean FARMER, Ft. Lauderdale, FL (US)

(21) Appl. No.: 17/770,173

(22) PCT Filed: Mar. 14, 2022
(86) PCT No.: PCT/US2022/020157

§ 371 Date: Sep. 22, 2022

Related U.S. Application Data
(60) Provisional application No. 63/161,154, filed on Mar. 15, 2021.

(51) Int. Cl. A01N 63/22; A01N 25/12; A01N 63/20; A01N 63/32; A01N 63/38; A01P 21/00; C09K 17/16

(52) U.S. Cl. CPC A01N 63/22; A01N 25/12; A01N 63/20; A01N 63/32; A01N 63/38; A01P 21/00; C09K 17/16

(57) ABSTRACT

The subject invention provides microbe-based agricultural compositions and methods of their use for preserving soil profiles and/or re-building degraded soil profiles, particularly for soil types that are prone to soil organic content (SOC) decomposition, oxidation and/or erosion. Advantageously, the compositions and methods of the subject invention are environmentally-friendly, non-toxic and cost effective solutions to the growing problem of soil degradation and soil-borne greenhouse gas emissions.

### CROSS-REFERENCE TO RELATED APPLICATION

This application claims priority to U.S. Provisional Patent Application No. 63/161,154, filed Mar. 15, 2021, which is incorporated herein by reference in its entirety.

### BACKGROUND OF THE INVENTION

Soil is a complex mixture of minerals, gases, liquids, organic matter and microorganisms. The specific composition of a particular type of soil varies based on factors such as, for example, human activity, geographic location and climate.

Muck soil, sometimes referred to as drained peat soil, is a type of soil found in temperate regions (e.g., Northern Europe and North America) and tropical regions (South Florida, South East Asia, South America, South Africa and the Caribbean) in areas where swamps, wetlands and/or other bodies of water have been drained over time.

More specifically, muck soils are defined by the USDA Natural Resources Conservation Service as sapric organic soils that are saturated more than 30 cumulative days in normal years or are artificially drained. Thus, these soils are made up mostly of humus from drained swamplands and other bodies of water (USDA 2014).

Muck soils can contain about 10 to 80% organic matter, compared with minerals soils, which contain about 1 to 5% organic matter.

Crops such as turf, onions, potatoes, radishes, carrots, celery and sugar cane are commonly grown in muck soils. Farming on muck lands is controversial, however, in part because it can entail the draining of wetlands and other wildlife habitats. Thus, environmental regulations will likely prevent the addition of new muck farm areas in the United States.

When cultivated, muck soils have a tendency to degrade over time (sometimes referred to as “subsidence”) as a result of draining, which leads to oxygenation of the soil environment and speeds up the aerobic breakdown of organic matter by soil microorganisms and/or their extracellular enzymes. Furthermore, when the soil surface is dry, the lightweight organic soil particles are readily eroded by wind (Warncke 2014).

The loss of soil profile in muck soil regions is a growing issue in areas that depend on the land for growing crops, such as, for example, southern Florida sugar cane growers. With the threat of total loss of cultivable muck soils, solutions are needed for preserving high organic content soils.

Additionally, as the amount of microbial decomposition of carbon-rich organic matter in soil increases, the result is an increase in the rate of atmospheric greenhouse gas (GHG) emissions, such as carbon dioxide, methane and nitrous oxide, from these processes and a decrease in the amount of soil organic carbon (SOC).

Soil organic carbon (SOC) is an important component of soil matter and consists mainly of plant and animal tissue remains, live microbial biomass, and the by-products of microbial processes, as well as organo-mineral complexes. As part of the broader carbon exchange cycle, even minor changes in SOC can have a large impact on the levels of atmospheric carbon dioxide in a region (1 Pg of soil carbon stock=0.47 ppm of atmospheric CO2).

Sequestration of SOC occurs when CO2 is transferred from the atmosphere into the soil by way of plant residues and other organic materials, which are stored in the soil with a long mean residence time (MRT). SOC sequestration can be achieved by, for example, increasing plant growth, retaining above and below-ground plant biomass, and/or protecting and stabilizing the SOC against erosion and decomposition.

A positive soil carbon budget is created by increasing the input of biomass carbon to exceed the SOC losses by erosion and decomposition. The rate of decomposition of biomass is affected by many factors, including, for example, climate, moisture levels, and types of plant matter—live or dead—present in the soils (Lal 2017).

An additional important factor impacting the rate of soil carbon accumulation is soil aggregate formation and stability. Healthy and robust root systems are effective for forming and stabilizing carbon-capturing soil aggregates, where organic matter and minerals become enmeshed in the roots. Soil microorganisms (e.g., fungal hyphae), and the growth by-products thereof (e.g., polysaccharides), can also facilitate association of carbon with soil mineral particles to form and stabilize these aggregates. Furthermore, studies have shown that the greater the soil aggregate size, the lower the degradation of soil by extracellular enzymes produced by microorganisms that consume organic matter in soil (Trivedi, P. et al. 2017; Trivedi, P. et al. 2015; Possinger et al. 2020; Grandy 2007).

The current belief in the industry is that the loss of muck and drained peat soils cannot be reversed or even mitigated, making muck soil a non-renewable resource. One thought is that the only way to reduce decomposition of muck soils and the resulting greenhouse gas emissions is to re-flood the soils; however, this does not address the issue of preserving muck/peaty soils for crop production, as most crops cannot grow in the flooded soils.

Other possible mitigation techniques include implementing no-till practices, shallow/modest-till practices, and/or increasing the use of off-season cover crops to reduce decomposition and erosion of soil organic matter. The theory is that such practices reduce the disturbance of larger carbon-rich soil aggregates, and aid in stabilizing these aggregates due to a consistent presence of plant root structures and the resulting presence of high carbon use efficiency (CUE) root-associating microbial populations in the soil (Grandy 2007; Panettieri et. al. 2012; Kallenbach, et. al. 2015; Kallenbach, et. al. 2019)

Combinations of these practices may help in preserving the soil profile; nonetheless, there is a need for enhancing the process further to help re-build and regenerate soil organic matter for the sake of ensuring the future of muck land farming, as well as reducing the emissions of atmospheric greenhouse gases from soils.

### BRIEF SUMMARY OF THE INVENTION

The subject invention provides microbe-based agricultural compositions and methods of their use for preserving soil profiles and/or re-building degraded soil profiles, particularly for soil types that are prone to soil organic content (SOC) decomposition, oxidation and/or erosion. Advantageously, the compositions and methods of the subject invention are environmentally-friendly, non-toxic and cost effective solutions to the growing problem of soil degradation and soil-borne greenhouse gas emissions.

In certain embodiments, the subject invention provides soil treatment compositions comprising one or more soil-colonizing microorganisms and/or growth by-products thereof, such as biosurfactants, enzymes and/or other metabolites. The composition may also comprise the fermentation medium in which the microorganism(s) were produced.

In certain embodiments, the microorganisms are bacteria, yeasts and/or fungi. In some embodiments, the composition comprises more than one type and/or species of microorganism. Advantageously, in some embodiments, the microorganisms colonize the rhizosphere and convert root exudates and digested organic matter into bulky, carbon-rich microbial biomass and necromass (dead cells). In some embodiments, the microorganisms form biofilms.

In preferred embodiments, the soil treatment compositions are utilized in methods for preserving, rebuilding and/or regenerating soils in need thereof. In certain preferred embodiments, the soil comprises at least 10% organic matter by volume, at least 50% organic matter, or at least 80% organic matter. In a specific embodiment, the soil is classified as muck soil, mucky peat and/or peat soil.

In certain embodiments, the methods comprise applying the soil treatment composition to soil in which a plant is, or will be, growing. The composition can be formulated for applying to soil and/or to above- and below-ground plant parts. For example, in certain embodiments, the composition can be mixed with water and applied to plants and/or to soil via an irrigation system.

In one embodiment, the soil treatment composition comprises a Bacillus sp. bacterium, such as, e.g., B. amyloliquefaciens NRRL B-67928. In one embodiment, the composition comprises a Trichoderma sp. fungus, such as, e.g., T. harzianum or T. guizhouse. In certain embodiments, the Bacillus sp. and the Trichoderma sp. are utilized together.

In one embodiment, the composition comprises one or more yeasts, such as, for example, Wickerhamomyces anomalus, Meyerozyma guilliermondii, Saccharomyces boulardii, Debaryomyces hansenii, Pichia occidentalis and/or Pichia kudriavzevii.

Advantageously, in some embodiments, the one or more microbes colonize the soil, as well as the plant roots, and aid in, for example, solubilizing nutrients for plant root uptake, dispersing water and salts throughout the rhizosphere, and/or increasing above and below-ground plant biomass, compared with untreated soils and/or plants.

In certain embodiments, the subject methods enhance SOC sequestration via, for example, increased above- and below-ground plant biomass, increased microbial biomass and/or necromass, and/or increased size and/or stability of soil aggregates. Furthermore, in certain embodiments, the methods can slow and/or stop soil profile degradation and/or erosion in areas where soil subsidence is occurring. Preferably, in some embodiments, the methods actually contribute to an increase in the depth of the soil profile.

Additionally, in certain embodiments, the subject methods can reduce the soil-borne emission of greenhouse gases, such as carbon dioxide, methane and nitrous oxide, which are caused by, for example, the decomposition of soil by low carbon use efficiency (CUE) microbes.

In certain embodiments, the methods of the subject invention further comprise applying one or more “accelerators” to the soil prior to, simultaneously with, and/or after the application of the soil treatment composition, such that the accelerator is available to the microorganisms of the soil treatment composition.

As used herein, an “accelerator” is any compound or substance that, when applied in the presence of the subject compositions, further decreases the rate of soil subsidence, increases the depth of the soil profile, increases the sequestration of SOC, enhances the health and/or growth of plant biomass, and/or decreases the rate of atmospheric carbon dioxide and other GHGs emitted from soil, compared to application of the soil treatment composition without the accelerator.

In one embodiment, the accelerator is a food source for the microorganisms. Non-limiting examples of food source accelerators include humic acid, kelp extract, fulvic acid, molasses, and mill mud.

In certain embodiments, the food source is one that is not typically found in the soil being treated, thereby providing a more diverse source of nutrients to the soil microorganisms. In some embodiments, the food source can be chosen based on the preferences of the particular microbe(s) in the soil treatment composition.

Advantageously, in some embodiments, the increased diversity of food resources encourages the diversification of the soil microbiome, which can lead to a reduction in the number of low CUE microorganisms microbes to decompose soil matter and produce GHGs. Additionally, in some embodiments, by increasing the availability of food resources for microbial consumption, the demand for carbon substrates is reduced, thereby reducing the production of enzymes that degrade labile carbon by soil microorganisms.

In one embodiment, the accelerator is a source of minerals and/or trace elements. In a specific embodiment, the minerals and/or trace elements are in the form of rock dust comprising finely crushed rock (also referred to as, e.g., rock minerals, rock flour, rock powder, stone dust, and/or mineral fines). Preferably, the rock dust is made up of basaltic rocks and/or silicate rocks that release elements such as calcium, magnesium, potassium, phosphorus and/or iron when weathering, or dissolving, in soil.

Advantageously, in some embodiments, the minerals and/or trace elements provide bioavailable micronutrients to enhance the health and/or growth of plants as well as microbes growing in the soil. In some embodiments, the minerals and/or trace elements facilitate the formation of carbon-mineral soil aggregates, the stability of which can be further enhanced by the microorganisms of the subject invention and/or plant root mass.

In some embodiments, the minerals and/or trace elements react with soil components to reduce carbon dioxide and/or nitrous oxide emissions from soil. For example, in one embodiment, the rock dust dissolves, reacting with carbon dioxide and capturing it in the form of carbon-storage molecules such as bicarbonates, calcium carbonate, and carbonate ions. In another embodiment, the rock dust alters the pH of the soil (e.g., increases it) as it weathers. Lower pH environments tend to inactivate diazotrophic N₂O reductase, which functions to reduce N₂O to N₂. Increasing the pH can allow for N₂O reductase to regain activity, thereby increasing the reduction of N₂O to N₂ and decreasing N₂O emissions.

In some embodiments, the subject methods also comprise performing one or more measurements to assess the effect of the methods of the subject invention on the generation and/or reduction in generation of GHGs, and/or the accumulation of carbon in soil. In one embodiment, the method comprises simply measuring the depth of the soil profile to determine whether the soil profile has decreased, increased, and/or remained stable after treatment with the subject compositions over time.

In some embodiments, the subject invention can be used for reducing the number of carbon credits used by an operator involved in, e.g., agriculture, livestock production, forestry/reforestation, and wetland management.

The methods and compositions of the subject invention can be used either alone or in combination with other compounds for efficiently enhancing soil and/or plant health. For example, in some embodiments, the method comprises applying additional components, such as herbicides, fertilizers, pesticides and/or other soil amendments, to the soil and/or plants. The exact materials and the quantities thereof can be determined by, for example, a grower or soil scientist having the benefit of the subject disclosure.

In some embodiments, the methods are used in combination with existing soil preservation practices, such as no-till or low-till farming, crop rotation, and/or the planting of off-season cover crops.

Advantageously, the subject compositions and methods can help re-build soil resources that are traditionally considered non-renewable, while suppressing and/or averting soil GHG emissions and reducing the need for synthetic fertilizers.

### DETAILED DESCRIPTION OF THE INVENTION

The subject invention provides microbe-based agricultural compositions and methods of their use for preserving soil profiles and/or re-building degraded soil profiles, particularly for soil types that are prone to soil organic content (SOC) decomposition, oxidation and/or erosion. Advantageously, the compositions and methods of the subject invention are environmentally-friendly, non-toxic and cost effective solutions to the growing problem of soil degradation and soil-borne greenhouse gas emissions.

### Selected Definitions

As used herein, “agriculture” means the cultivation and breeding of plants for food, fiber, biofuel, medicines, cosmetics, supplements, ornamental purposes and other uses. According to the subject invention, agriculture can also include horticulture, landscaping, gardening, plant conservation, forestry and reforestation, pasture and prairie restoration, orcharding, arboriculture, and agronomy. Further included in agriculture are the care, monitoring and maintenance of soil.

As used herein, a “broth,” “culture broth,” or “fermentation broth” refers to a culture medium comprising at least nutrients and microorganism cells.

As used herein, the term “carbon use efficiency” or “CUE” refers to a generalized measure of the efficiency by which microbes allocate carbon taken up towards growth and biomass production versus respiration. CUE can be calculated as growth (biomass production) over the sum of CO₂ production/emissions and growth. Microorganisms are often categorized as “low CUE” or “high CUE,” where a CUE greater than 0.50 is considered high, and a CUE lower than 0.50 is considered low.

Unless the context requires otherwise, the phrases “fermenting,” “fermentation process” or “fermentation reaction” and the like, as used herein, are intended to encompass both the growth phase and product biosynthesis phase of the process.

As used herein, an “isolated” or “purified” compound is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. “Isolated” in the context of a microbial strain means that the strain is removed from the environment in which it exists in nature. Thus, the isolated strain may exist as, for example, a biologically pure culture, or as spores (or other forms of the strain) in association with a carrier.

As used herein, a “biologically pure culture” is a culture that has been isolated from materials with which it is associated in nature. In a preferred embodiment, the culture has been isolated from all other living cells. In further preferred embodiments, the biologically pure culture has advantageous characteristics compared to a culture of the same microbe as it exists in nature. The advantageous characteristics can be, for example, enhanced production of one or more growth by-products.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 85%, 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, “enhancing” means improving or increasing. For example, enhanced plant health means improving the plant's ability grow and thrive, which includes increased seed germination and/or emergence, improved immunity against pests and/or diseases, and improved ability to survive environmental stressors, such as droughts and/or overwatering. Enhanced plant growth and/or enhanced plant biomass means increasing the size and/or mass of a plant above and/or below the ground (e.g., increased canopy/foliar volume, height, trunk caliper, branch length, shoot length, protein content, root size/density and/or overall growth index), and/or improving the ability of the plant to reach a desired size and/or mass. Enhanced yields mean improving the end products produced by the plants in a crop, for example, by increasing the number and/or size of fruits, leaves, roots and/or tubers per plant, and/or improving the quality of the fruits, leaves, roots and/or tubers (e.g., improving taste, texture, brix, chlorophyll content and/or color).

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites include, but are not limited to, biosurfactants, biopolymers, enzymes, acids, solvents, alcohols, proteins, vitamins, minerals, microelements, and amino acids.

The subject invention utilizes “microbe-based compositions,” meaning a composition that comprises components that were produced as the result of the growth of microorganisms or other cell cultures. Thus, the microbe-based composition may comprise the microbes themselves and/or by-products of microbial growth. The microbes may be in a vegetative state, in spore or conidia form, in hyphae form, in any other form of propagule, or a mixture of these. The microbes may be planktonic or in a biofilm form, or a mixture of both. The by-products of growth may be, for example, metabolites, cell membrane components, proteins, and/or other cellular components. The microbes may be intact or lysed. In preferred embodiments, the microbes are present, with growth medium in which they were grown, in the microbe-based composition. The microbes may be present at, for example, a concentration of at least 1×10⁴, 1×10⁵, 1×10⁶, 1×10⁷, 1×10⁸, 1×10⁹, 1×10¹⁰, 1×10¹¹, 1×10¹² or 1×10¹³ or more CFU per gram or per ml of the composition.

The subject invention further provides “microbe-based products,” which are products that are to be applied in practice to achieve a desired result. The microbe-based product can be simply a microbe-based composition harvested from a microbe cultivation process. Alternatively, the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein “preventing” or “prevention” of a situation or occurrence means delaying, inhibiting, suppressing, forestalling, and/or minimizing the onset, extensiveness or progression of the situation or occurrence. Prevention can include, but does not require, indefinite, absolute or complete prevention, meaning it may still develop at a later time. Prevention can include reducing the severity of the onset of such a situation or occurrence, and/or stalling its development to a more severe or extensive situation or occurrence.

Ranges provided herein are understood to be shorthand for all of the values within the range. For example, a range of 1 to 20 is understood to include any number, combination of numbers, or sub-range from the group consisting of 1, 2, 3, 4, 5, 6, 7, 8, 9, 10, 11, 12, 13, 14, 15, 16, 17, 18, 19, 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduction” refers to a negative alteration, and the term “increase” refers to a positive alteration, wherein the negative or positive alteration is at least 0.25%, 0.5%, 1%, 5%, 10%, 15%, 20%, 25%, 30%, 35%, 40%, 45%, 50%, 55%, 60%, 70%, 75%, 80%, 85%, 90%, 95%, or 100%.

As used herein, “reference” refers to a standard or control condition.

As used herein, a “soil amendment” or a “soil conditioner” is any compound, material, or combination of compounds or materials that are added into soil to enhance the properties of the soil and/or rhizosphere. Soil amendments can include organic and inorganic matter, and can further include, for example, fertilizers, pesticides and/or herbicides. Nutrient-rich, well-draining soil is essential for the growth and health of plants, and thus, soil amendments can be used for enhancing the plant biomass by altering the nutrient and moisture content of soil. Soil amendments can also be used for improving many different qualities of soil, including but not limited to, soil structure (e.g., preventing compaction); improving the nutrient concentration and storage capabilities; improving water retention in dry soils; and improving drainage in waterlogged soils.

As used herein, “surfactant” refers to a compound that lowers the surface tension (or interfacial tension) between phases. Surfactants act as, e.g., detergents, wetting agents, emulsifiers, foaming agents, and dispersants. A “biosurfactant” is a surfactant produced by a living organism.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof. All references cited herein are hereby incorporated by reference in their entirety.

### Methods for Enhancing Degraded Soils

The subject invention provides microbe-based agricultural compositions and methods of their use for preserving soil profiles and/or re-building degraded soil profiles, particularly for soil types that are prone to soil organic content (SOC) decomposition, oxidation and/or erosion. Advantageously, the compositions and methods of the subject invention are environmentally-friendly, non-toxic and cost effective solutions to the growing problem of soil degradation and soil-borne greenhouse gas emissions.

In certain embodiments, the subject invention provides soil treatment compositions comprising one or more soil-colonizing microorganisms and/or growth by-products thereof, such as biosurfactants, enzymes, polysaccharides and/or other metabolites. The composition may also comprise the fermentation medium in which the microorganism(s) were produced.

In certain embodiments, the microorganisms are bacteria, yeasts and/or fungi. In some embodiments, the composition comprises more than one type and/or species of microorganism. Advantageously, in some embodiments, the microorganisms colonize the rhizosphere and convert root exudates and digested organic matter into bulky, carbon-rich microbial biomass and necromass (dead cells).

In preferred embodiments, the soil treatment compositions are utilized in methods for preserving, rebuilding and/or regenerating soils in need thereof. In certain preferred embodiments, the soil comprises at least 10%, at least 25%, at least 50%, at least 75% or at least 80% organic matter by volume. In a specific embodiment, the soil is classified as muck soil, mucky peat and/or peat soil.

In certain embodiments, the methods comprise applying the soil treatment composition to soil in which a plant is, or will be, growing.

In certain embodiments, the one or more microorganisms colonize the soil and/or roots of the plants, and provide one or more benefits to the plants that result in enhanced utilization and/or storage of carbon via enhanced growth and/or health of both aerial and subterranean plant tissue. For example, the microorganisms and their growth by-products can aid in solubilizing nutrients for plant root uptake, as well as dispersing water and salts throughout the rhizosphere, compared with untreated soils and/or plants.

In some embodiments, the subject methods increase the above- and below-ground biomass of plants, which includes, for example, increased foliage volume, increased stem and/or trunk diameter, enhanced root growth and/or density, and/or increased total numbers of plants. In one embodiment, this is achieved by improving the overall hospitability of the rhizosphere in which a plant's roots are growing, for example, by improving the nutrient and/or moisture retention properties of the rhizosphere. In one embodiment, the soil treatment compositions enhance penetration of beneficial molecules through the outer layers of root cells, for example, at the root-soil interface of the rhizosphere.

In one embodiment, the composition can lead to improved biodiversity of the soil microbiome. As used herein, improving the biodiversity refers to increasing the variety of microbial species within the soil. In some embodiments, improved biodiversity comprises increasing the ratio of high CUE microorganisms to low CUE microorganisms, and/or converting low CUE microorganisms into high CUE microorganisms.

In certain embodiments, the subject methods enhance SOC sequestration via increased soil microbial biomass and/or necromass, and/or increased size and/or stability of soil aggregates. Thus, in certain embodiments, the methods can slow and/or stop soil profile degradation and/or erosion in areas where soil subsidence is occurring. Preferably, in certain embodiments, the methods actually contribute to an increase in the depth of the soil profile.

Additionally, in certain embodiments, the subject methods can reduce the soil-borne emission of greenhouse gases, such as carbon dioxide, methane and nitrous oxide, which are caused by, for example, the decomposition of soil by low CUE microbes.

In certain embodiments, the methods of the subject invention further comprise applying one or more “accelerators” prior to, simultaneously with, and/or after the application of the soil treatment composition, such that the accelerator is available to the microorganisms of the soil treatment composition.

As used herein, an “accelerator” is any compound or substance that, when applied in the presence of the subject compositions, further decreases the rate of soil subsidence, increases the depth of the soil profile, increases the sequestration of SOC, enhances the health and/or growth of plant biomass, and/or decreases the rate of atmospheric carbon dioxide and other GHGs emitted from soil, compared to application of the soil treatment composition without the accelerator.

In one embodiment, the accelerator is a food source for the microorganisms. Non-limiting examples of food source accelerators include humic acid, kelp extract, fulvic acid, molasses, and mill mud.

In certain embodiments, the food source is one that is not typically found in the soil being treated, thereby providing a more diverse source of nutrients to the soil microorganisms. In some embodiments, the food source can be chosen based on the preferences of the particular microbe(s) in the soil treatment composition.

Advantageously, in some embodiments, the increased diversity of food resources encourages the diversification of the soil microbiome, which can lead to a reduction in the number of low CUE microorganisms and/or methanogenic microbes to decompose soil matter and produce GHGs, such as carbon dioxide and methane. Additionally, in some embodiments, by increasing the availability of food resources for microbial consumption, the demand for carbon substrates is reduced, thereby reducing the production of enzymes that degrade labile carbon by soil microorganisms.

In some embodiments, the lower CUE microbes are converted into higher CUE due to the greater availability of nutrients.

In one embodiment, the accelerator is a source of minerals and/or trace elements. In a specific embodiment, the minerals and/or trace elements are in the form of rock dust comprising finely crushed rock (also referred to as, e.g., rock minerals, rock flour, rock powder, stone dust, and/or mineral fines). In certain embodiments, the particle size on application is about 5 to 100 um, or about 8 to 80 um, or about 10 to 50 um, or about 12 to 30 um.

The rock dust can be applied at a rate of, for example, 0.1 to 10 tons/acre per year, or 0.2 to 9 tons/acre per year, or 0.3 to 8 tons/acre per year, or 0.4 to 7 tons/acre per year, or 0.5 to 6 tons/acre per year, or 0.6 to 5 tons/acre per year, or 0.7 to 4 tons/acre per year, or 0.8 to 3 tons/acre per year, or 0.9 to 2 tons/acre per year, or 1 to 1.5 tons/acre per year.

Preferably, the rock dust is made up of basaltic rocks, limestones, and/or silicate rocks that release elements such as calcium, magnesium, potassium, phosphorus and/or iron when weathering, or dissolving, in soil.

More specifically, in certain embodiments, the rock dust comprises silicate rocks such as, e.g., olivine, garnet, zircon, wollastonite, calcium silicate, epidot, melilite, tourmaline, pyroxene, amphibole, micas, clays, quartz, feldspars, and/or zeolites.

In certain embodiments, the rock dust comprises igneous basaltic rocks and/or limestones.

Advantageously, in some embodiments, the minerals and/or trace elements provide bioavailable micronutrients, such as, e.g., magnesium, phosphate, nitrogen, potassium, selenium, calcium, sulfur, iron, copper, and zinc, to enhance the health and/or growth of plants as well as microbes growing in the soil. In some embodiments, the minerals and/or trace elements facilitate the formation of carbon-mineral soil aggregates, the stability of which can be further enhanced by the microorganisms of the subject invention and/or plant root mass.

In some embodiments, the minerals and/or trace elements react with soil components to reduce carbon dioxide and/or nitrous oxide emissions from soil. For example, in one embodiment, the rock dust dissolves and releases readily water-soluble cations such as calcium, sodium and magnesium. In certain embodiments, these cations bind CO₂ from the atmosphere and/or from soil, forming carbon-storage molecules such as bicarbonates, calcium carbonate, and carbonate ions and trapping carbon in the soil.

In another embodiment, the weathering of rock dust and release of cations alters the pH of the soil (i.e., increases it) as it weathers. Lower pH environments tend to inactivate diazotrophic N₂O reductase, which functions to reduce N₂O to N₂. Increasing the pH can allow for N₂O reductase to regain activity, thereby increasing the reduction of N₂O to N₂ and decreasing N₂O emissions.

According to the subject methods, the soil treatment compositions, and if applicable, accelerator(s), can be used either alone or in combination with other compounds for efficiently enhancing soil and/or plant health. For example, in some embodiments, the method comprises applying additional components, such as herbicides, fertilizers, pesticides and/or other soil amendments, to the soil and/or plants. The exact materials and the quantities thereof can be determined by, for example, a grower or soil scientist having the benefit of the subject disclosure.

In some embodiments, prior to applying a composition to the site, the method comprises assessing the site for local conditions, determining a preferred formulation for the composition (e.g., the type, combination and/or ratios of microorganisms and/or growth by-products) that is customized for the local conditions, and producing the composition with the preferred formulation.

The local conditions can include, for example, soil conditions (e.g., soil type, species of soil microbiota, amount and/or type of soil organic content, amount and/or type of GHG precursor substrates, amount and/or type of fertilizers or other soil additives or amendments present); crop and/or plant conditions (e.g., types, numbers, age and/or health of plants being grown); environmental conditions (e.g., current climate, season, or time of year); amount and type of GHG emissions at the site; mode and/or rate of application of the composition, and others as are relevant to the site.

After assessment, a preferred formulation for the composition can be determined so that the composition can be customized for these local conditions. The composition is then cultivated, preferably at a microbe growth facility that is within 300 miles of the site of application, preferably within 200 miles, even more preferably within 100 miles.

In some embodiments the local conditions are assessed periodically, for example, once annually, biannually, or even monthly. In this way, the composition formula can be modified in real time as necessary to meet the unique needs of the changing local conditions.

In some embodiments, the subject methods also comprise performing one or more measurements to assess the effect of the methods of the subject invention on the generation and/or reduction in generation of GHGs, and/or the accumulation of carbon in soil. In one embodiment, the method comprises simply measuring the depth of the soil profile to determine whether the soil profile has decreased, increased, and/or remained stable after treatment with the subject compositions over time.

In certain embodiments, the subject methods also comprise performing one or more measurements to assess the effect of the methods of the subject invention on the generation and/or reduction in generation of GHGs and/or on the accumulation of SOC in plants and/or soil.

Measurements can be conducted at a certain time point after application of the soil treatment composition to the site. In some embodiments, the measurements are conducted after about 1 week or less, 2 weeks or less, 3 weeks or less, 4 weeks or less, 30 days or less, 60 days or less, 90 days or less, 120 days or less, 180 days or less, and/or 1 year or less.

Furthermore, the measurements can be repeated over time. In some embodiments, the measurements are repeated daily, weekly, monthly, bi-monthly, semi-monthly, semi-annually, and/or annually.

In certain embodiments, assessing GHG generation can take the form of measuring GHG emissions from a site. Gas chromatography with electron capture detection is commonly used for testing samples in a lab setting. In certain embodiments, GHG emissions can also be conducted in the field, using, for example, flux measurements and/or in situ soil probing. Flux measurements analyze the emission of gases from the soil surface to the atmosphere, for example, using chambers that enclose an area of soil and then estimate flux by observing the accumulation of gases inside the chamber over a period of time. Probes can be used to generate a soil gas profile, starting with a measurement of the concentration of the gases of interest at a certain depth in the soil, and comparing it directly between probes and ambient surface conditions (Brummell and Siciliano 2011, at 118).

Measuring GHG emissions can also comprise other forms of direct emissions measurement, gas chromatography-mass spectrometry (GC-MS) and/or analysis of fuel input. Direct emissions measurements can comprise, for example, identifying polluting operational activities (e.g., fuel-burning automobiles) and measuring the emissions of those activities directly through Continuous Emissions Monitoring Systems (GEMS). Fuel input analysis can comprise calculating the quantity of energy resources used (e.g., amount of electricity, fuel, wood, biomass, etc., consumed) determining the content of, for example, carbon, in the fuel source, and applying that carbon content to the quantity of the fuel consumed to determine the amount of emissions.

In certain embodiments, carbon content of a site where plants are growing, e.g., agricultural site, crop, sod or turf farm, pasture/prairie or forest, can be measured by, for example, quantifying the aboveground and/or below-ground biomass of plants. In general, the carbon concentration of, for example, a tree, is assumed to be from about 40 to 50% of the biomass.

Biomass quantification can take the form of, for example, harvesting plants in a sample area and measuring the weight of the different parts of the plant before and after drying. Biomass quantification can also be carried out using non-destructive, observational methods, such as measuring, e.g., trunk diameter, height, volume, and other physical parameters of the plant. Remote quantification can also be used, such as, for example, laser profiling and/or drone analysis.

In some embodiments, carbon content of a site can further comprise sampling and measuring carbon content of litter, woody debris and/or soil of a sampling area. Soil, in particular, can be analyzed, for example, using dry combustion to determine percent total organic carbon (TOC); by potassium permanganate oxidation analysis for detecting active carbon; and by bulk density measurements (weight per unit volume) for converting from percent carbon to tons/acre.

In some embodiments, the subject invention can be used for reducing the number of carbon credits used by an operator involved in, e.g., agriculture, livestock production, forestry/reforestation, and wetland management.

The methods and compositions of the subject invention can be used either alone or in combination with other compounds for efficiently enhancing soil and/or plant health. For example, in some embodiments, the method comprises applying additional components, such as herbicides, fertilizers, pesticides and/or other soil amendments, to the soil and/or plants. The exact materials and the quantities thereof can be determined by, for example, a grower or soil scientist having the benefit of the subject disclosure.

In some embodiments, the methods are used in combination with existing soil preservation practices, such as no-till or low-till farming, crop rotation, and/or the planting of off-season cover crops.

Advantageously, the subject compositions and methods can help re-build soil resources that are traditionally considered non-renewable, while suppressing and/or averting soil GHG emissions and reducing the need for synthetic fertilizers.

### Modes of Application

As used herein, “applying” a composition or product to a site refers to contacting a composition or product with a site such that the composition or product can have an effect on that site. The effect can be due to, for example, microbial growth and colonization, and/or the action of a metabolite, enzyme, biosurfactant or other microbial growth by-product, and/or activity of an accelerator substance. The mode of application depends upon the formulation of the composition, and can include, for example, spraying, pouring, sprinkling, injecting, spreading, mixing, dunking, fogging and misting. Formulations can include, for example, liquids, dry and/or wettable powders, flowable powders, dusts, granules, pellets, emulsions, microcapsules, steaks, oils, gels, pastes and/or aerosols. In an exemplary embodiment, the subject soil treatment composition is applied after the composition has been prepared by, for example, dissolving the composition in water.

In one embodiment, the site to which the composition is applied is the soil (or rhizosphere) in which plants will be planted or are growing (e.g., a crop, a field, an orchard, a grove, a pasture/prairie or a forest). The compositions of the subject invention can be pre-mixed with irrigation fluids, wherein the compositions percolate through the soil and can be delivered to, for example, the roots of plants to influence the root microbiome.

In one embodiment, the compositions are applied to soil surfaces, with or without water, where the beneficial effect of the soil application can be activated by rainfall, sprinkler, flood, or drip irrigation.

In one embodiment, the site is a plant or plant part. The composition can be applied directly thereto as a seed treatment, or to the surface of a plant or plant part (e.g., to the surface of the roots, tubers, stems, flowers, leaves, fruit, or flowers). In a specific embodiment, the composition is contacted with one or more roots of the plant. The composition can be applied directly to the roots, e.g., by spraying or dunking the roots, and/or indirectly, e.g., by administering the composition to the soil in which the plant grows (or the rhizosphere). The composition can be applied to the seeds of the plant prior to or at the time of planting, or to any other part of the plant and/or its surrounding environment.

In one embodiment, wherein the method is used in a large scale setting, such as in a muck field, a sugarcane crop, a citrus grove, a pasture or prairie, a forest, a sod or turf farm, or another agricultural crop, the method can comprise administering the composition into a tank connected to an irrigation system used for supplying water, fertilizers, pesticides or other liquid compositions. Thus, the plant and/or soil surrounding the plant can be treated with the composition via, for example, soil injection, soil drenching, using a center pivot irrigation system, with a spray over the seed furrow, with micro-jets, with drench sprayers, with boom sprayers, with sprinklers and/or with drip irrigators. Advantageously, the method is suitable for treating hundreds or more acres of land.

In one embodiment, wherein the method is used in a smaller scale setting, the method can comprise pouring the composition (mixed with water and other optional additives) into the tank of a handheld lawn and garden sprayer and spraying soil or another site with the composition. The composition can also be mixed into a standard handheld watering can and poured onto a site.

Soil, plants and/or their environments can be treated at any point during the process of cultivating the plant. For example, the composition can be applied to the soil prior to, concurrently with, or after the time when seeds or plants are planted therein. It can also be applied at any point thereafter during the development and growth of the plant, including when the plant is flowering, fruiting, and during and/or after abscission of leaves.

In one embodiment, the methods and compositions according to the subject invention lead to an increase in one or more of: root mass, stalk diameter, plant height, canopy density, chlorophyll content, flower count, bud count, bud size, bud density, leaf surface area, and/or nutrient uptake of a plant, by at least 1%, 5%, 10%, 20%, 30%, 40%, 50%, 60%, 70%, 80%, 90%, 100%, 150%, 200%, or more, compared to a plant growing in an untreated environment.

In one embodiment, the methods and compositions according to the subject invention cause an increase in SOC in an area of soil, by at least 1%, 5%, 10%, 20%, 30%, 40%, 50%, 60%, 70%, 80%, 90%, 100%, 150%, 200%, or more, compared to similar untreated areas.

In one embodiment, the methods and compositions according to the subject invention cause an increase in depth of soil profile by at least 1%, 5%, 10%, 20%, 30%, 40%, 50%, 60%, 70%, 80%, 90%, 100%, 150%, 200%, or more, compared to similar untreated areas.

In one embodiment, the methods and compositions according to the subject invention cause a decrease in soil-borne emissions of GHG, such as CO2, N2O and/or CH4, by at least 1%, 5%, 10%, 20%, 30%, 40%, 50%, 60%, 70%, 80%, 90%, 100%, or more, compared to similar untreated soil.

### Target Plants

As used here, the term “plant” includes, but is not limited to, any species of woody, ornamental or decorative, crop or cereal, fruit plant or vegetable plant, flower or tree, macroalga or microalga, phytoplankton and photosynthetic algae (e.g., green algae Chlamydomonas reinhardtii). “Plant” also includes a unicellular plant (e.g. microalga) and a plurality of plant cells that are largely differentiated into a colony (e.g. volvox) or a structure that is present at any stage of a plant's development. Such structures include, but are not limited to, a fruit, a seed, a shoot, a stem, a leaf, a root, a flower petal, etc. Plants can be standing alone, for example, in a garden, or can be one of many plants, for example, as part of an orchard, crop or pasture.

As used herein, “crop plants” refer to any species of plant or alga, grown for profit and/or for sustenance for humans, animals or aquatic organisms, or used by humans (e.g., textile, cosmetics, and/or drug production), or viewed by humans for pleasure (e.g., flowers or shrubs in landscaping or gardens) or any plant or alga, or a part thereof, used in industry, commerce or education. Crop plants can be plants that can be obtained by traditional breeding and optimization methods or by biotechnological and recombinant methods, or combinations of these methods, including the transgenic plants and the plant varieties.

Types of crop plants that can benefit from application of the products and methods of the subject invention include, but are not limited to: row crops (e.g., corn, soy, sorghum, peanuts, potatoes, etc.), field crops (e.g., alfalfa, wheat, grains, etc.), tree crops (e.g., walnuts, almonds, pecans, hazelnuts, pistachios, etc.), citrus crops (e.g., orange, lemon, grapefruit, etc.), fruit crops (e.g., apples, pears, strawberries, blueberries, blackberries, etc.), turf crops (e.g., sod), ornamentals crops (e.g., flowers, vines, etc.), vegetables (e.g., tomatoes, carrots, etc.), vine crops (e.g., grapes, etc.), forestry (e.g., pine, spruce, eucalyptus, poplar, etc.), managed pastures (any mix of plants used to support grazing animals).

Additional examples of plants for which the subject invention is useful include, but are not limited to, cereals and grasses (e.g., wheat, barley, rye, oats, rice, maize, sorghum, corn), beets (e.g., sugar or fodder beets); fruit (e.g., grapes, strawberries, raspberries, blackberries, pomaceous fruit, stone fruit, soft fruit, apples, pears, plums, peaches, almonds, cherries or berries); leguminous crops (e.g., beans, lentils, peas or soya); oil crops (e.g., oilseed rape, mustard, poppies, olives, sunflowers, coconut, castor, cocoa or ground nuts); cucurbits (e.g., pumpkins, cucumbers, squash or melons); fiber plants (e.g., cotton, flax, hemp or jute); citrus fruit (e.g., oranges, lemons, grapefruit or tangerines); vegetables (e.g., spinach, lettuce, asparagus, cabbages, carrots, onions, tomatoes, potatoes or bell peppers); Lauraceae (e.g., avocado, Cinnamonium or camphor); and also tobacco, nuts, herbs, spices, medicinal plants, coffee, eggplants, sugarcane, tea, pepper, grapevines, hops, the plantain family, latex plants, cut flowers and ornamentals.

In certain embodiments, the crop plant is a citrus plant. Examples of citrus plants according to the subject invention include, but are not limited to, orange trees, lemon trees, lime trees and grapefruit trees. Other examples include Citrus maxima (Pomelo), Citrus medica (Citron), Citrus micrantha (Papeda), Citrus reticulata (Mandarin orange), Citrus paradisi (grapefruit), Citrus japonica (kumquat), Citrus australasica (Australian Finger Lime), Citrus australis (Australian Round lime), Citrus glauca (Australian Desert Lime), Citrus garrawayae (Mount White Lime), Citrus gracilis (Kakadu Lime or Humpty Doo Lime), Citrus inodora (Russel River Lime), Citrus warburgiana (New Guinea Wild Lime), Citrus wintersii (Brown River Finger Lime), Citrus halimii (limau kadangsa, limau kedut kera), Citrus indica (Indian wild orange), Citrus macroptera, and Citrus latipes, Citrus x aurantiifolia (Key lime), Citrus x aurantium (Bitter orange), Citrus x latifolia (Persian lime), Citrus x limon (Lemon), Citrus x limonia (Rangpur), Citrus x sinensis (Sweet orange), Citrus x tangerina (Tangerine), Imperial lemon, tangelo, orangelo, tangor, kinnow, kiyomi, Minneola tangelo, oroblanco, ugli, Buddha's hand, citron, bergamot orange, blood orange, calamondin, clementine, Meyer lemon, and yuzu.

In some embodiments, the crop plant is a relative of a citrus plant, such as orange jasmine, limeberry, and trifoliate orange (Citrus trifolata).

Additional examples of target plants include all plants that belong to the superfamily Viridiplantae, in particular monocotyledonous and dicotyledonous plants including fodder or forage legumes, ornamental plants, food crops, trees or shrubs selected from Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g., A. sativa, A. fatua, A. byzantina, A. fatua var. sativa, A. hybrida), Averrhoa carambola, Bambusa sp., Benincasa hispida, Bertholletia excelsea, Beta vulgaris, Brassica spp. (e.g., B. napus, B. rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g., E. guineensis, E. oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g., G. max, Soja hispida or Soja max), Gossypium hirsutum, Helianthus spp. (e.g., H. annuus), Hemerocallis fulva, Hibiscus spp., Hordeum spp. (e.g., H. vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g., L. esculentum, L. lycopersicum, L. pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g., O. sativa, O. latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp. (e.g., Q. suber L), Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g., S. tuberosum, S. integrifolium or S. lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g., T. aestivum, T. durum, T. turgidum, T. hybernum, T. macha, T. sativum, T. monococcum or T. vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

Target plants can also include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Target vegetable plants include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the embodiments include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Plants of the embodiments include crop plants (for example, corn, alfalfa, sunflower, Brassica, soybean, cotton, safflower, peanut, sorghum, wheat, millet, tobacco, etc.), such as corn and soybean plants.

Target turfgrasses include, but are not limited to: annual bluegrass (Poa annua); annual ryegrass (Lolium multiflorum); Canada bluegrass (Poa compressa); Chewings fescue (Festuca rubra); colonial bentgrass (Agrostis tenuis); creeping bentgrass (Agrostis palustris); crested wheatgrass (Agropyron desertorum); fairway wheatgrass (Agropyron cristatum); hard fescue (Festuca longifolia); Kentucky bluegrass (Poa pratensis); orchardgrass (Dactylis glomerate); perennial ryegrass (Lolium perenne); red fescue (Festuca rubra); redtop (Agrostis alba); rough bluegrass (Poa trivialis); sheep fescue (Festuca ovine); smooth bromegrass (Bromus inermis); tall fescue (Festuca arundinacea); timothy (Phleum pretense); velvet bentgrass (Agrostis canine); weeping alkaligrass (Puccinellia distans); western wheatgrass (Agropyron smithii); Bermuda grass (Cynodon spp.); St. Augustine grass (Stenotaphrum secundatum); zoysia grass (Zoysia spp.); Bahia grass (Paspalum notatum); carpet grass (Axonopus affinis); centipede grass (Eremochloa ophiuroides); kikuyu grass (Pennisetum clandesinum); seashore paspalum (Paspalum vaginatum); blue gramma (Bouteloua gracilis); buffalo grass (Buchloe dactyloids); sideoats gramma (Bouteloua curtipendula).

Further plants of interest include grain plants that provide seeds of interest, oil-seed plants, and leguminous plants. Seeds of interest include grain seeds, such as corn, wheat, barley, rice, sorghum, rye, millet, etc. Oil-seed plants include cotton, soybean, safflower, sunflower, Brassica, maize, alfalfa, palm, coconut, flax, castor, olive etc. Leguminous plants include beans and peas. Beans include guar, locust bean, fenugreek, soybean, garden beans, cowpea, mungbean, lima bean, fava bean, lentils, chickpea, etc. Further plants of interest include Cannabis (e.g., sativa, indica, and ruderalis) and industrial hemp.

In certain specific embodiments, the plant is one typically grown in muck soils, muck peat soil, peat soil and/or drained peat soil, such as, for example, sugar cane, potatoes, onion, celery, carrot, radish, and turf.

All plants and plant parts can be treated in accordance with the invention. In this context, plants are understood as meaning all plants and plant populations such as desired and undesired wild plants or crop plants (including naturally occurring crop plants). Crop plants can be plants that can be obtained by traditional breeding and optimization methods or by biotechnological and recombinant methods, or combinations of these methods, including the transgenic plants and the plant varieties.

Plant tissue and/or plant parts are understood as meaning all aerial and subterranean parts and organs of the plants such as shoots, leaves, flowers, roots, needles, stalks, stems, fruits, seeds, tubers and rhizomes. The plant parts also include crop material and vegetative and generative propagation material, for example cuttings, tubers, rhizomes, slips and seeds.

### Soil Treatment Compositions

In certain embodiments, the subject invention provides soil treatment compositions comprising one or more soil-colonizing microorganisms and/or growth by-products thereof, such as biosurfactants, enzymes, polysaccharides and/or other metabolites. The composition may also comprise the fermentation broth/medium in which the microorganism(s) were produced.

In some embodiments, the microorganisms of the subject invention have a greater CUE greater than microbes already present in the soil to which they are applied. In some embodiments, the microorganisms of the subject composition are “high CUE,” meaning the percentage of carbon they allocate to biomass production is greater than the percentage allocated to respiration.

In certain embodiments, the microorganisms are bacteria, yeasts and/or fungi. In some embodiments, the composition comprises more than one type and/or species of microorganism. Advantageously, in some embodiments, the microorganisms colonize the rhizosphere and convert root exudates and digested organic matter into bulky, carbon-rich microbial biomass and necromass (dead cells).

In preferred embodiments, the microbe-based compositions according to the subject invention are non-toxic and can be applied in high concentrations without causing irritation to, for example, the skin or digestive tract of a human or other non-pest animal. Thus, the subject invention is particularly useful where application of the microbe-based compositions occurs in the presence of living organisms, such as growers and livestock.

In one embodiment, multiple microorganisms can be used together, where the microorganisms create a synergistic benefit towards plant and root health, as well as increasing SOC, preventing soil degradation and/or rebuilding degraded soils.

The species and ratio of microorganisms and other ingredients in the composition can be customized and optimized for specific local conditions at the time of application, such as, for example, which soil type, plant and/or crop is being treated; what season, climate and/or time of year it is when a composition is being applied; and what mode and/or rate of application is being utilized. Thus, the composition can be customizable for any given site.

The microorganisms useful according to the subject invention can be, for example, non-plant-pathogenic strains of bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the microorganism is a yeast or fungus. Yeast and fungus species suitable for use according to the current invention, include Aureobasidium (e.g., A. pullulans), Blakeslea, Candida (e.g., C. apicola, C. bombicola, C. nodaensis), Cryptococcus, Debaryomyces (e.g., D. hansenii), Entomophthora, Hanseniaspora, (e.g., H. uvarum), Hansenula, Issatchenkia, Kluyveromyces (e.g., K. phaffii), Lentinula edodes, Mortierella, mycorrhizal fungi, Meyerozyma (M. guilliermondii, M. aphidis), Penicillium, Phycomyces, Pichia (e.g., P. anomala, P. guilliermondii, P. occidentalis, P. kudriavzevii), Pleurotus spp. (e.g., P. ostreatus), Pseudozyma (e.g., P. aphidis), Saccharomyces (e.g., S. boulardii sequela, S. cerevisiae, S. torula), Starmerella (e.g., S. bombicola), Torulopsis, Trichoderma (e.g., T. guizhouse, T. reesei, T. harzianum, T. koningii, T. hamatum, T. viride), Ustilago (e.g., U. maydis), Wickerhamomyces (e.g., W. anomalus), Williopsis (e.g., W. mrakii), Zygosaccharomyces (e.g., Z. bailii), and others.

In certain embodiments, the microorganisms are bacteria, including Gram-positive and Gram-negative bacteria. The bacteria may be, for example Agrobacterium (e.g., A. radiobacter), Azotobacter (A. vinelandii, A. chroococcum), Azospirillum (e.g., A. brasiliensis), Bacillus (e.g., B. amyloliquefaciens, B. circulans, B. firmus, B. laterosporus, B. licheniformis, B. megaterium, B. mucilaginosus, B. polymyxa, B. subtilis (including strains B1, B2, B3 and B4), Brevibacillus laterosporus), Frateuria (e.g., F. aurantia), Microbacterium (e.g., M. laevaniformans), myxobacteria (e.g., Myxococcus xanthus, Stignatella aurantiaca, Sorangium cellulosum, Minicystis rosea), Paenibacillus polymyxa, Pantoea (e.g., P. agglomerans), Pseudomonas (e.g., P. aeruginosa, P. chlororaphis subsp. aureofaciens (Kluyver), P. putida), Rhizobium spp., Rhodospirillum (e.g., R. rubrum), Sphingomonas (e.g., S. paucimobilis), and/or Thiobacillus thiooxidans (Acidothiobacillus thiooxidans).

In certain embodiments, the microorganism is one that is capable of fixing and/or solubilizing nitrogen, potassium, phosphorous and/or other micronutrients in soil.

In one embodiment, the microorganism is a nitrogen-fixing microorganism, or a diazotroph, selected from species of, for example, Azospirillum, Azotobacter, Chlorobiaceae, Cyanothece, Frankia, Klebsiella, rhizobia, Trichodesmium, and some Archaea. In a specific embodiment, the nitrogen-fixing bacteria is Azotobacter vinelandii.

In one embodiment, the microorganism is a potassium-mobilizing microorganism, or KMB, selected from, for example, Bacillus mucilaginosus, Frateuria aurantia or Glomus mosseae. In a specific embodiment, the potassium-mobilizing microorganism is Frateuria aurantia.

In one embodiment, the microorganism is a non-denitrifying microorganism capable of converting nitrous oxide from the atmosphere into nitrogen in the soil, such as, for example, Dyadobacter fermenters.

In one embodiment, a combination of microorganisms is used in the subject microbe-based composition, wherein the microorganisms work synergistically with one another to enhance plant biomass, and/or to enhance the properties of the rhizosphere.

In specific exemplary embodiments, the microbes utilized according to the subject invention are selected from one or more of: Trichoderma spp. (e.g., T. harzianum, T viride, T. koningii, and T. guizhouse); Bacillus spp. (e.g., B. amyloliquefaciens, B. subtilis, B. megaterium, B. polymyxa, B. licheniformis, and Brevibacillus laterosporus); Meyerozyma guilliermondii; Pichia occidentalis; Pichia kudriavzevii; Wickerhamomyces anomalus; and Debaryomyces hansenii.

In another specific exemplary embodiment, the composition comprises Meyerozyma guilliermondii or Meyerozyma caribbica (e.g., M. caribbica MEC14XN).

In another specific exemplary embodiment, the composition comprises a Bacillus bacterium, such as B. amyloliquefaciens and/or B. subtilis.

In another specific exemplary embodiment, the composition comprises B. amyloliquefaciens NRRL B-67928 and a Trichoderma sp., such as, for example, T. harzianum (e.g., T. harzianum T-22).

In one embodiment, the composition comprises B. amyloliquefaciens NRRL B-67928 “B. amy.” A culture of the B. amyloliquefaciens “B. amy” microbe has been deposited with the Agricultural Research Service Northern Regional Research Laboratory (NRRL) Culture Collection, 1815 N. University St., Peoria, IL, USA. The deposit has been assigned accession number NRRL B-67928 by the depository and was deposited on Feb. 26, 2020.

In one embodiment, the composition comprises B. subtilis NRRL B-68031 “B4.” A culture of the B4 microbe has been deposited with the Agricultural Research Service Northern Regional Research Laboratory (NRRL) Culture Collection, 1815 N. University St., Peoria, IL, USA. The deposit has been assigned accession number NRRL B-68031 by the depository and was deposited on May 6, 2021.

In one embodiment, the composition comprises W. anomalus NRRL Y-68030. A culture of this microbe has been deposited with the Agricultural Research Service Northern Regional Research Laboratory (NRRL) Culture Collection, 1815 N. University St., Peoria, IL, USA. The deposit has been assigned accession number NRRL Y-68030 by the depository and was deposited on May 6, 2021.

The subject culture has been deposited under conditions that assure that access to the culture will be available during the pendency of this patent application to one determined by the Commissioner of Patents and Trademarks to be entitled thereto under 37 CFR 1.14 and 35 U.S.C 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

In a specific embodiment, the concentration of each microorganism included in the composition is 1×10⁶ to 1×10¹³ CFU/g, 1×10⁷ to 1×10¹² CFU/g, 1×10⁸ to 1×10¹¹ CFU/g, or 1×10⁹ to 1×10¹⁰ CFU/g of the composition.

In one embodiment, the total microbial cell concentration of the composition is at least 1×10⁶ CFU/g, including up to 1×10⁹ CFU/g, 1×10¹⁰, 1×10¹¹, 1×10¹² and/or 1×10¹³ or more CFU/g. In one embodiment, the microorganisms of the subject composition comprise about 5 to 20% of the total composition by weight, or about 8 to 15%, or about 10 to 12%.

The composition can comprise the leftover fermentation substrate and/or purified or unpurified growth by-products, such as enzymes, biosurfactants and/or other metabolites. The microbes can be live or inactive.

The microbes and microbe-based compositions of the subject invention have a number of beneficial properties that are useful for, e.g., increasing plant biomass and/or forming/stabilizing carbo-mineral soil aggregates. For example, the compositions can comprise products resulting from the growth of the microorganisms, such as biosurfactants, proteins and/or enzymes, either in purified or crude form. Furthermore, the microorganisms can enhance plant growth, induce auxin production, enable solubilization, absorption and/or balance of nutrients in the soil, and protect plants from pests and pathogens.

In one embodiment, the microorganisms of the subject composition are capable of producing a biosurfactant. In another embodiment, biosurfactants can be produced separately by other microorganisms and added to the composition, either in purified form or in crude form. Crude form biosurfactants can comprise, for example, biosurfactants and other products of cellular growth in the leftover fermentation medium resulting from cultivation of a biosurfactant-producing microbe. This crude form biosurfactant composition can comprise from about 0.001% to about 90%, about 25% to about 75%, about 30% to about 70%, about 35% to about 65%, about 40% to about 60%, about 45% to about 55%, or about 50% pure biosurfactant.

Biosurfactants form an important class of secondary metabolites produced by a variety of microorganisms such as bacteria, fungi, and yeasts. As amphiphilic molecules, microbial biosurfactants reduce the surface and interfacial tensions between the molecules of liquids, solids, and gases. Furthermore, the biosurfactants according to the subject invention are biodegradable, have low toxicity, are effective in solubilizing and degrading insoluble compounds in soil and can be produced using low cost and renewable resources. They can inhibit adhesion of undesirable microorganisms to a variety of surfaces, prevent the formation of biofilms, and can have powerful emulsifying and demulsifying properties. Furthermore, the biosurfactants can also be used to improve wettability and to achieve even solubilization and/or distribution of fertilizers, nutrients, and water in the soil.

Biosurfactants according to the subject methods can be selected from, for example, low molecular weight glycolipids (e.g., sophorolipids, cellobiose lipids, rhamnolipids, mannosylerythritol lipids and trehalose lipids), lipopeptides (e.g., surfactin, iturin, fengycin, arthrofactin and lichenysin), flavolipids, phospholipids (e.g., cardiolipins), fatty acid esters, and high molecular weight polymers such as lipoproteins, lipopolysaccharide-protein complexes, and polysaccharide-protein-fatty acid complexes.

The composition can comprise one or more biosurfactants at a concentration of 0.001% to 10%, 0.01% to 5%, 0.05% to 2%, and/or from 0.1% to 1% by weight.

The composition can comprise the fermentation medium containing a live and/or an inactive culture, the purified or crude form growth by-products, such as biosurfactants, enzymes, and/or other metabolites, and/or any residual nutrients.

The product of fermentation may be used directly, with or without extraction or purification. If desired, extraction and purification can be easily achieved using standard extraction and/or purification methods or techniques described in the literature.

The microorganisms in the composition may be in an active or inactive form, or in the form of vegetative cells, reproductive spores, mycelia, hyphae, conidia or any other form of microbial propagule. The composition may also contain a combination of any of these microbial forms.

In one embodiment, when a combination of strains of microorganism are included in the composition, the different strains of microbe are grown separately and then mixed together to produce the composition.

Advantageously, in accordance with the subject invention, the composition may comprise the medium in which the microbes were grown. The composition may be, for example, at least, by weight, 1%, 5%, 10%, 25%, 50%, 75%, or 100% growth medium. The amount of biomass in the composition, by weight, may be, for example, anywhere from 0% to 100% inclusive of all percentages therebetween.

In one embodiment, the composition is preferably formulated for application to soil, seeds, whole plants, or plant parts (including, but not limited to, roots, tubers, stems, flowers and leaves). In certain embodiments, the composition is formulated as, for example, liquid, dust, granules, microgranules, pellets, wettable powder, flowable powder, emulsions, microcapsules, oils, or aerosols.

To improve or stabilize the effects of the composition, it can be blended with suitable adjuvants and then used as such or after dilution, if necessary. In preferred embodiments, the composition is formulated as a liquid, a concentrated liquid, or as dry powder or granules that can be mixed with water and other components to form a liquid product. In one embodiment, the composition can comprise glucose (e.g., in the form of molasses), in addition to an osmoticum substance, to ensure optimum osmotic pressure during storage and transport of the dry product.

Further components can be added to the composition, for example, buffering agents, carriers, other microbe-based compositions produced at the same or different facility, viscosity modifiers, preservatives, nutrients for microbe growth, tracking agents, biocides, other microbes, surfactants, emulsifying agents, lubricants, solubility controlling agents, pH adjusting agents, preservatives, stabilizers and ultra-violet light resistant agents.

The pH of the composition should be suitable for the microorganism of interest as well as for the soil environment to which it will be applied. In some embodiments, the pH is about 2.0 to about 10.0, about 2.0 to about 9.5, about 2.0 to about 9.0, about 2.0 to about 8.5, about 2.0 to about 8.0, about 2.0 to about 7.5, about 2.0 to about 7.0, about 3.0 to about 7.5, about 4.0 to about 7.5, about 5.0 to about 7.5, about 5.5 to about 7.0, about 6.5 to about 7.5, about 3.0 to about 5.5, about 3.25 to about 4.0, or about 3.5. Buffers, and pH regulators, such as carbonates and phosphates, may be used to stabilize pH near a preferred value.

Optionally, the composition can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

The microbe-based compositions may be used without further stabilization, preservation, and storage, however. Advantageously, direct usage of these microbe-based compositions preserves a high viability of the microorganisms, reduces the possibility of contamination from foreign agents and undesirable microorganisms, and maintains the activity of the by-products of microbial growth.

In other embodiments, the composition (microbes, growth medium, or microbes and medium) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 pint to 1,000 gallons or more. In certain embodiments the containers are 1 gallon, 2 gallons, 5 gallons, 25 gallons, or larger.

The compositions can be used in combination with other agricultural compounds and/or crop management systems. In one embodiment, the composition can optionally comprise, or be applied with, for example, natural and/or chemical pesticides, repellants, herbicides, fertilizers, water treatments, non-ionic surfactants and/or soil amendments. Preferably, however, the composition does not comprise and/or is not used with benomyl, dodecyl dimethyl ammonium chloride, hydrogen dioxide/peroxyacetic acid, imazilil, propiconazole, tebuconazole, or triflumizole.

If the composition is mixed with compatible chemical additives, the chemicals are preferably diluted with water prior to addition of the subject composition.

In one embodiment, the subject compositions are compatible for use with agricultural compounds characterized as antiscalants, such as, e.g., hydroxyethylidene diphosphonic acid;

bactericides, such as, e.g., streptomycin sulfate and/or Galltrol® (A. radiobacter strain K84);

biocides, such as, e.g., chlorine dioxide, didecyldimethyl ammonium chloride, halogenated heterocyclic, and/or hydrogen dioxide/peroxyacetic acid;

fertilizers, such as, e.g., N-P-K fertilizers, calcium ammonium nitrate 17-0-0, potassium thiosulfate, nitrogen (e.g., 10-34-0, Kugler KQ-XRN, Kugler KS-178C, Kugler KS-2075, Kugler LS 6-24-6S, UN 28, UN 32), and/or potassium;

fungicides, such as, e.g., chlorothalonil, manicozeb hexamethylenetetramine, aluminum tris, azoxystrobin, Bacillus spp. (e.g., B. licheniformis strain 3086, B. subtilis, B. subtilis strain QST 713), benomyl, boscalid, pyraclostrobin, captan, carboxin, chloroneb, chlorothalonil, copper culfate, cyazofamid, dicloran, dimethomorph, etridiazole, thiophanate-methyl, fenamidone, fenarimol, fludioxonil, fluopicolide, flutolanil, iprodione, mancozeb, maneb, mefanoxam, fludioxonil, mefenoxam, metalaxyl, myclobutanil, oxathiapiprolin, pentachloronitrobenzene (quintozene), phosphorus acid, propamocarb, propanil, pyraclostrobin, Reynoutria sachalinensis, Streptomyces spp. (e.g., S. griseoviridis strain K61, S. lydicus WYEC 108), sulfur, urea, thiabendazole, thiophanate methyl, thiram, triadimefon, triadimenol, and/or vinclozolin;

growth regulators, such as, e.g., ancymidol, chlormequat chloride, diaminozide, paclobutrazol, and/or uniconazole;

herbicides, such as, e.g., glyphosate, oxyfluorfen, and/or pendimethalin;

insecticides, such as, e.g., acephate, azadirachtin, B. thuringiensis (e.g., subsp. israelensis strain AM 65-52), Beauveria bassiana (e.g., strain GHA), carbaryl, chlorpyrifos, cyantraniliprole, cyromazine, dicofol, diazinon, dinotefuran, imidacloprid, Isaria fumosorosae (e.g., Apopka strain 97), lindane, and/or malathion;

water treatments, such as, e.g., hydrogen peroxide (30-35%), phosphonic acid (5-20%), and/or sodium chlorite;

as well as glycolipids, lipopeptides, deet, diatomaceous earth, citronella, essential oils, mineral oils, garlic extract, chili extract, and/or any known commercial and/or homemade pesticide that is determined to be compatible by the skilled artisan having the benefit of the subject disclosure.

Preferably, the composition does not comprise and/or is not applied simultaneously with, or within 7 to 10 days before or after, application of the following compounds: benomyl, dodecyl dimethyl ammonium chloride, hydrogen dioxide/peroxyacetic acid, imazilil, propiconazole, tebuconazole, or triflumizole.

In certain embodiments, the compositions and methods can be used to enhance the effectiveness of other compounds, for example, by enhancing the penetration of a pesticidal compound into a plant or pest, or enhancing the bioavailability of a nutrient to plant roots. The microbe-based products can also be used to supplement other treatments, for example, antibiotic treatments. Advantageously, the subject invention helps reduce the amount of antibiotics that must be administered to a crop or plant in order to be effective at treating and/or preventing bacterial infection.

### Growth of Microbes According to the Subject Invention

The subject invention utilizes methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

As used herein “fermentation” refers to cultivation or growth of cells under controlled conditions. The growth could be aerobic or anaerobic. In preferred embodiments, the microorganisms are grown using SSF and/or modified versions thereof.

In one embodiment, the subject invention provides materials and methods for the production of biomass (e.g., viable cellular material), extracellular metabolites (e.g. small molecules and proteins), residual nutrients and/or intracellular components (e.g. enzymes and other proteins).

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of organisms in a sample. The technique can also provide an index by which different environments or treatments can be compared.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source can be a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sunflower oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In some embodiments, the method for cultivation may further comprise adding additional acids and/or antimicrobials in the medium before, and/or during the cultivation process. Antimicrobial agents or antibiotics are used for protecting the culture against contamination.

Additionally, antifoaming agents may also be added to prevent the formation and/or accumulation of foam during submerged cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

The pH of the culture should be suitable for the microorganism of interest as well as for the soil environment to which the composition will be applied. In some embodiments, the pH is about 2.0 to about 10.0, about 2.0 to about 9.5, about 2.0 to about 9.0, about 2.0 to about 8.5, about 2.0 to about 8.0, about 2.0 to about 7.5, about 2.0 to about 7.0, about 3.0 to about 7.5, about 4.0 to about 7.5, about 5.0 to about 7.5, about 5.5 to about 7.0, about 6.5 to about 7.5, about 3.0 to about 5.5, about 3.25 to about 4.0, or about 3.5. Buffers, and pH regulators, such as carbonates and phosphates, may be used to stabilize pH near a preferred value.

In one embodiment, the method of cultivation is carried out at about 5° to about 100° C., about 15° to about 60° C., about 20° to about 50° C., about 20° to about 45° C., about 25° to about 40° C., about 25° to about 37° C., about 25° to about 35° C., about 30° to about 35° C., about 24° to about 28° C., or about 22° to about 25° C. In one embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

The microbial growth by-product produced by microorganisms of interest may be retained in the microorganisms or secreted into the growth medium. The medium may contain compounds that stabilize the activity of microbial growth by-product.

The biomass content of the fermentation medium may be, for example, from 5 g/l to 180 g/l or more, or from 10 g/l to 150 g/l.

The cell concentration may be, for example, at least 1×10⁶ to 1×10¹³, 1×10⁷ to 1×10¹², 1×10⁸ to 1×10¹¹, or 1×10⁹ to 1×10¹⁰ CFU/ml.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

Advantageously, the microbe-based products can be produced in remote locations. The microbe growth facilities may operate off the grid by utilizing, for example, solar, wind and/or hydroelectric power.

### Preparation of Microbe-Based Products

One microbe-based product of the subject invention is simply the fermentation medium containing the microorganisms and/or the microbial metabolites produced by the microorganisms and/or any residual nutrients. The product of fermentation may be used directly without extraction or purification. If desired, extraction and purification can be easily achieved using standard extraction and/or purification methods or techniques described in the literature.

The microorganisms in the microbe-based products may be in an active or inactive form, or in the form of vegetative cells, reproductive spores, conidia, mycelia, hyphae, or any other form of microbial propagule. The microbe-based products may also contain a combination of any of these forms of a microorganism.

In one embodiment, different strains of microbe are grown separately and then mixed together to produce the microbe-based product. The microbes can, optionally, be blended with the medium in which they are grown and dried prior to mixing.

In one embodiment, the different strains are not mixed together, but are applied to a plant and/or its environment as separate microbe-based products.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

Upon harvesting the microbe-based composition from the growth vessels, further components can be added as the harvested product is placed into containers or otherwise transported for use. The additives can be, for example, buffers, carriers, other microbe-based compositions produced at the same or different facility, viscosity modifiers, preservatives, nutrients for microbe growth, surfactants, emulsifying agents, lubricants, solubility controlling agents, tracking agents, solvents, biocides, antibiotics, pH adjusting agents, chelators, stabilizers, ultra-violet light resistant agents, other microbes and other suitable additives that are customarily used for such preparations.

In one embodiment, buffering agents including organic and amino acids or their salts, can be added. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

In one embodiment, additional components such as an aqueous preparation of a salt, such as sodium bicarbonate or carbonate, sodium sulfate, sodium phosphate, sodium biphosphate, can be included in the formulation.

In certain embodiments, an adherent substance can be added to the composition to prolong the adherence of the product to plant parts. Polymers, such as charged polymers, or polysaccharide-based substances can be used, for example, xanthan gum, guar gum, levan, xylinan, gellan gum, curdlan, pullulan, dextran and others.

In preferred embodiments, commercial grade xanthan gum is used as the adherent. The concentration of the gum should be selected based on the content of the gum in the commercial product. If the xanthan gum is highly pure, then 0.001% (w/v—xanthan gum/solution) is sufficient.

In one embodiment, glucose, glycerol and/or glycerin can be added to the microbe-based product to serve as, for example, an osmoticum during storage and transport. In one embodiment, molasses can be included.

In one embodiment, prebiotics can be added to and/or applied concurrently with the microbe-based product to enhance microbial growth. Suitable prebiotics, include, for example, kelp extract, fulvic acid, chitin, humate and/or humic acid. In a specific embodiment, the amount of prebiotics applied is about 0.1 L/acre to about 0.5 L/acre, or about 0.2 L/acre to about 0.4 L/acre.

In one embodiment, specific nutrients are added to and/or applied concurrently with the microbe-based product to enhance microbial inoculation and growth. These can include, for example, soluble potash (K₂O), magnesium, sulfur, boron, iron, manganese, and/or zinc. The nutrients can be derived from, for example, potassium hydroxide, magnesium sulfate, boric acid, ferrous sulfate, manganese sulfate, and/or zinc sulfate.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used (e.g., a citrus grove). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This allows for a scaled-down bioreactor (e.g., smaller fermentation vessel, smaller supplies of starter material, nutrients and pH control agents), which makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

In one embodiment, the microbe growth facility is located on, or near, a site where the microbe-based products will be used (e.g., a citrus grove), for example, within 300 miles, 200 miles, or even within 100 miles. Advantageously, this allows for the compositions to be tailored for use at a specified location. The formula and potency of microbe-based compositions can be customized for specific local conditions at the time of application, such as, for example, which soil type, plant and/or crop is being treated; what season, climate and/or time of year it is when a composition is being applied; and what mode and/or rate of application is being utilized.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products to improve GHG management.

The cultivation time for the individual vessels may be, for example, from 1 to 7 days or longer. The cultivation product can be harvested in any of a number of different ways.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Compositions

Exemplified herein is a composition according to certain embodiments of subject invention for use in reducing GHGs, improving carbon utilization, and/or enhancing sequestration of carbon. This example is not to be intended as limiting. Formulations comprising other species of microorganisms, either in lieu of, or in addition to, those exemplified here, may be included in the composition.

The composition comprises a microbial inoculant comprising a Trichoderma spp. fungus and a Bacillus spp. bacterium. In specific instances, the composition comprises Trichoderma harzianum and Bacillus amyloliquefaciens. Even more specifically, the strain of B. amyloliquefaciens can be B. amyloliquefaciens NRRL B-67928.

In one embodiment, the composition can comprise from 1 to 99% Trichoderma by weight and from 99 to 1% Bacillus by weight. In some embodiments, the cell count ratio of Trichoderma to Bacillus is about 1:9 to about 9:1, about 1:8 to about 8:1, about 1:7 to about 7:1, about 1:6 to about 6:1, about 1:5 to about 5:1 or about 1:4 to about 4:1.

The composition can comprise about 1×10⁶ to 1×10¹², 1×10⁷ to 1×10¹¹, 1×10⁸ to 1×10¹⁰, or 1×10⁹ CFU/ml of the Trichoderma; and about 1×10⁶ to 1×10¹², 1×10⁷ to 1×10¹¹, 1×10⁸ to 1×10¹⁰, or 1×10⁹ CFU/ml of the Bacillus.

The composition can be mixed with and/or applied concurrently with additional “starter” materials to promote initial growth of the microorganisms in the composition. These can include, for example, prebiotics and/or nano-fertilizers (e.g., Aqua-Yield, NanoGro™).

One exemplary formulation of such growth-promoting “starter” materials comprises one or more of:
Soluble potash (K2O) (1.0% to 2.5%, or about 2.0%)
Magnesium (Mg) (0.25% to 0.75%, or about 0.5%)
Sulfur (S) (2.5% to 3.0%, or about 2.7%)
Boron (B) (0.01% to 0.05%, or about 0.02%)
Iron (Fe) (0.25% to 0.75%, or about 0.5%)
Manganese (Mn) (0.25% to 0.75%, or about 0.5%)
Zinc (Zn) (0.25% to 0.75%, or about 0.5%)
Humic acid (8% to 12%, or about 10%)
Kelp extract (5% to 10%, or about 6%)
Water (70% to 85%, or about 77% to 80%)

The microbial inoculant, and/or optional growth-promoting “starter” materials, are mixed with water in an irrigation system tank and applied to soil.

In specific instances, the composition comprises 10.0% by weight of the microbial inoculant, and 90% by weight water, where the inoculant comprises 1×10⁸ CFU/mL Trichoderma harzianum and 1×10⁹ CFU/mL of Bacillus amyloliquefaciens.

### Example 2—Microbial Strains

The subject invention utilizes beneficial microbial strains. In certain embodiments, the microorganism is a strain of Trichoderma, such as, e.g., a strain of T. harzianum, T. viride, T. longibrachia, T. asperellum, T. hamatum, T. koningii, T. reesei, T. guizhouse and/or others.

Exemplary Trichoderma harzianum strains can include, but are not limited to, T-315 (ATCC 20671); T-35 (ATCC 20691); 1295-7 (ATCC 20846); 1295-22 [T-22] (ATCC 20847); 1295-74 (ATCC 20848); 1295-106 (ATCC 20873); T12 (ATCC 56678); WT-6 (ATCC 52443): Rifa T-77 (CMI CC 333646); T-95 (60850); T12m (ATCC 20737); SK-55 (No. 13327; BP 4326 NIBH (Japan)); RR17Bc (ATCC PTA 9708); TSHTH20-1 (ATCC PTA 10317); AB 63-3 (ATCC 18647); OMZ 779 (ATCC 201359); WC 47695 (ATCC 201575); m5 (ATCC 201645); (ATCC 204065); UPM-29 (ATCC 204075); T-39 (EPA 119200); and/or F11Bab (ATCC PTA 9709).

In some embodiments, the microbe is a Bacillus strain, such as, e.g., B. subtilis, B. amyloliquefaciens, B. licheniformis, B. megaterium, B. polymyxa and/or others.

B. subtilis strains can include, e.g., B. subtilis B1 (ATCC PTA-123459), B2, B3 and/or B4 (NRRL B-68031).

Bacillus amyloliquefaciens strains can include, but are not limited to, NRRL B-67928, FZB24 (EPA 72098-5; BGSC 10A6), TA208, NJN-6, N2-4, N3-8, and those having ATCC accession numbers 23842, 23844, 23843, 23845, 23350 (strain DSM 7), 27505, 31592, 49763, 53495, 700385, BAA-390, PTA-7544, PTA-7545, PTA-7546, PTA-7549, PTA-7791, PTA-5819, PTA-7542, PTA-7790, and/or PTA-7541.

### REFERENCES

Brummell, M. E., and S. D. Siciliano. (2011). “Measurement of Carbon Dioxide, Methane, Nitrous Oxide, and Water Potential in Soil Ecosystems.” Methods in Enzymology. 496:115-137. Doi: 10.1016/B978-0-12-386489-5.00005-1. (“Brummell and Siciliano 2011”).
Grandy, A. S. and G. P. Robertson (2007). “Land-Use Intensity Effects on Soil Organic Carbon Accumulation Rates and Mechanisms.” Ecosystems 10:58-73. (“Grandy 2007”).
Kallenbach, C. M. et al. (2015). “Microbial physiology and necromass regulate agricultural soil carbon accumulation.” Soil Biol & Biochem 91:279-290. (“Kallenbach 2015”).
Kallenbach, C. M. et al. (2019). “Managing Agroecosystems for Soil Microbial Carbon Use Efficiency: Ecological Unknowns, Potential Outcomes, and a Path Forward.” Frontiers in Microbiol 10:1146. (“Kallenbach 2019”).
Panettieri, M. et al. (2013). “Moldboard plowing effects on soil aggregation and soil organic matter quality assessed by 13C CPMAS NMR and biochemical analyses.” Agric., Ecosys & Envt 177:48-57. (“Panettieri 2013”).
Possinger, A. R. et al. (2020). “Organo-organic and organo-mineral interfaces in soil at the nanometer scale.” Nature comm. 11:6103. (“Possinger 2020”).
Soil Survey Staff, USDA (2014). “Keys to Soil Taxonomy.” USDA Natural Resources Conservation Service. 12^{th} Edition. (“USDA 2014”).
Trivedi, P. et al. (2015). “Soil aggregate size mediates the impacts of cropping regimes on soil carbon and microbial communities.” Soil Biol & Biochem 91:169-181. (“Trivedi 2015”).
Trivedi, P. et al. (2017). “Soil aggregation and associated microbial communities modify the impact of agricultural management on carbon content.” Envtl Microbiol 19(8), 3070-3086. (“Trivedi 2017”).
Warncke, D. D., (2014). “Managing Muck Soils for Vegetable Crops.” Soil Fertility and Plant Nutrition, Michigan State University. (“Warncke 2014”). http://www.hort.cornell.edu/expo/proceedings/2014/Cover%20Crops%20Tillage%20and%20Soils/Muck%20Soils%20Warncke.pdf

1. A method for preventing the degradation of soil and/or for re-building degraded soil, which comprises applying a soil treatment composition comprising one or more beneficial microorganisms and/or one or more microbial growth by-products to a plant and/or to soil such that the one or more microorganisms colonize the plant's roots and/or the soil, wherein the beneficial microorganisms are bacteria, yeasts and/or fungi.

2. The method of claim 1, wherein the composition comprises a beneficial microorganism selected from Trichoderma harzianum, Trichoderma viride, Trichoderma koningii, Trichoderma guizhouse, Bacillus amyloliquefaciens, Bacillus subtilis, Bacillus megaterium, Bacillus polymyxa, Bacillus licheniformis, Brevibacillus laterosporus, Meyerozyma guilliermondii, Meyerozyma caribbica, Pichia occidentalis, Pichia kudriavzevii, Wickerhamomyces anomalus, and Debaryomyces hansenii.

3. The method of claim 1, further comprising applying an accelerator with the soil treatment composition, wherein the accelerator is a microbial food source or a source of minerals and/or trace elements.

4. The method of claim 3, wherein the microbial food source is selected from humic acid, kelp extract, fulvic acid, mill mud and molasses, and wherein the source of minerals and/or trace elements is silicate, basaltic or limestone rock dust.

5. The method of claim 1, wherein the composition comprises beneficial microorganisms Bacillus amyloliquefaciens NRRL B-67928 and Trichoderma harzianum.

6. The method of claim 1, wherein the composition comprises beneficial microorganisms selected from B. amyloliquefaciens NRRL B-67928, B. subtilis NRRL B-68031, and W. anomalus NRRL Y-68030.

7. (canceled)

8. The method of claim 1, wherein the soil treatment composition is applied to the plant and/or to the soil using an irrigation system.

9. The method of claim 1, wherein the soil has an organic matter content of at least 10%.

10. The method of claim 1, wherein the soil is muck soil or drained peat soil.

11. The method of claim 1, wherein, as a result of application of the soil treatment composition, one or more of the following occurs:
the soil organic content (SOC) of the soil is increased;
the rate of decomposition of SOC into atmospheric greenhouse gases by soil microorganisms is decreased;
the rate of soil profile loss is decreased; and
soil profile loss is reversed such that an increase in soil profile depth occurs.

12. The method of claim 11, wherein application of the soil treatment composition causes the increased SOC, decreased rate of SOC decomposition, decreased rate of soil profile loss and/or reversed soil profile loss by facilitating one or more of the following in the soil:
increased plant root biomass, increased microbial biomass, increased microbial necromass, and increased size and/or stability of soil organo-mineral aggregates.

13. The method of claim 11, further comprising performing a measurement to assess the effect of the method on increasing SOC, decreasing the rate of SOC decomposition, decreasing the rate of soil profile loss and/or reversing soil profile loss.

14. A soil treatment composition comprising one or more beneficial microorganisms selected from Bacillus amyloliquefaciens strain NRRL B-67928, Wickerhamomyces anomalus NRRL Y-68031 and B. subtilis NRRL B-68030.

15. (canceled)

16. The composition of claim 15, comprising Bacillus amyloliquefaciens strain NRRL B-67928.

17. (canceled)

18. The composition of claim 14, comprising a cell count ratio of 1:4, Trichoderma sp. to Bacillus amyloliquefaciens NRRL B-67928.

19. The composition of claim 14, comprising Wickerhamomyces anomalus NRRL Y-68031.

20. The composition of claim 14, further comprising Meyerozyma guilliermondii and/or Meyerozyma caribbica

21. The composition of claim 14, comprising B. subtilis NRRL B-68030.

22. The composition of claim 14, further comprising a microbial food source selected from kelp extract, fulvic acid, humate, humic acid, molasses and mill mud.

23. The composition of claim 14, further comprising silicate, basaltic and/or limestone rock dust.

24. The composition of claim 14, formulated as a dry powder or dry granules.
